(19) United States
(12) Patent Application Publication
(10) Pub. No.: US 2017/0138825 A1
(43) Pub. Date: May 18, 2017

(54) SYSTEM AND METHOD FOR TRACE SAMPLE PRECOLLECTION AND PRECONCENTRATION

(71) Applicant: TRACETECH SECURITY LTD., Tel Aviv (IL)

(72) Inventor: Fredy ORNATH, Tel Aviv (IL)

(73) Assignee: TRACETECH SECURITY LTD., Tel Aviv (IL)

(21) Appl. No.: 15/420,686

(22) Filed: Jan. 31, 2017

Related U.S. Application Data
(62) Division of application No. 14/759,140, filed on Jul. 2, 2015.
(60) Provisional application No. 61/748,455, filed on Jan. 3, 2013.
(60) Provisional application No. 61/748,456, filed on Jan. 3, 2013.

(51) Int. Cl. G01N 1/22; G01N 33/00; G01N 1/40

(52) U.S. Cl. CPC G01N 1/2226; G01N 1/405; G01N 33/0057; G01N 1/40; G01N 2001/4038

(57) ABSTRACT

A device and procedure are described that can be used for improved sampling of traces such as for explosive trace screening, by precollection and preconcentration of trace samples of vapor and particulate matter from air. The device is unique in its ability to collect both solid and vapor traces.

This nonprovisional application is a divisional of U.S. application Ser. No. 14/759,140, which was filed on Jul. 2, 2015, which was a continuation of International Application No. PCT/IB2013/002880, which was filed on December 29, 2013, and which claims priority to U.S. Provisional Application No. 61/748,455, which was filed on Jan. 3, 2013 and to U.S. Provisional Application No. 61/748,456, which was filed on Jan. 3, 2013, and which are all herein incorporated by reference.

### BACKGROUND

1. Technical Field

Embodiments of the present invention relate generally to systems and methods for trace sampling precollection and preconcentration as well as detection screening of packed or enclosed cargo, baggage, and unpacked cargo

2. Description of Related Art

Screening baggage and cargo items for the presence of explosives is a common procedure in transportation security procedures, homeland security standards and other situations. The present claimed invention is related with screening items (including packaged and/or wrapped objects) for substances of interest using trace detection by collecting traces of vapor and particles extant around substances of interest, due to evaporation, diffusion and the like.

As will be apparent to one skilled in the art, screening baggage and cargo for the presence of explosives, contraband, etc. is a common procedure in transportation security procedures, Homeland Security and other situations. Other applications may include quality assurance of agricultural products, pharma, chemical and bio threats, etc.

Explosive detection often employs the ubiquitous and nearly inevitable diffusion of vapor and/or particles from materials to locate and identify said materials, but also due to contamination by touching or friction during preparations. After collection, traces are analyzed, in principle allowing for use of the full arsenal of modern analytic chemistry including, MS, GC, IMS and the like. Bespoke detectors have been designed to detect and identify the presence of specific compounds such as explosive substances in trace amounts.

Vapor will in most cases form in air volumes around substances of interest due to the aforementioned processes of evaporation and diffusion, not to mention sublimation. This will occur to varying extents in keeping with volatility or vapor pressure of the substance(s) involved. Some substances have very low vapor pressures, and while vapor is always formed to an extent increasing with exposure time and temperature, the quantity of evaporated material can even so be exceedingly small and very difficult to detect and identify. For such materials it may be the case that detection is more tractable by detection of minute solid (or liquid) particles that are dispelled from surfaces due to mechanical forces such as vibration, friction, etc. Such particles, once free from the surface, will tend to disperse by means of Brownian motion, diffusion, and ambient air currents. Since the particles are often of extremely low weight and potentially high surface areas, they may float for long times before settling in absence of air currents or float for extended periods of time in the presence of sufficient air currents; in presence of currents they may in some cases float indefinitely depending on several parameters, for example particle size.

As expounded above, both vapor and particles of various substances of interest (such as drugs, explosives, radioactive materials, poisons, microorganisms, rare elements, and the like) can form as contamination during concealment of explosives in IEDs (improvised explosive device) that terrorists may prepare and that security agencies are tasked with intercepting.

### Explosive Trace Detection (ETD)

Explosive trace detection (ETD) exploits the aforementioned omnipresent vapor and/or particle diffusion from explosives to locate and foil the surreptitious use thereof by terrorists, smugglers, or other inimical entities. After collection, traces may be analyzed by any of the known ETD methods, which employ explosive trace detectors (aka electronic trace detectors) designed to discern and identify the presence of explosive substances even in minute, trace quantities.

### Trace Collection

Efficient collection of traces, also known as “sampling”, is critical for the success of the screening procedure, since even as the detector (ETD) can be extremely sensitive, its success is still heavily dependent on the quantity of sample brought to its nose. Thus the concept of sampling efficiency is central in the field of ETD; this refers to the amount of trace actually brought to a detector as a fraction of the entire amount of trace passing through the sampling device. The more efficient the sampling system, the larger a fraction of the entirety of extant traces are available for analysis by the detection device and means at hand, hence allowing detection of extremely dilute materials and/or reduction of sampling volume required and/or increase of sensitivity.

### Other Uses

Trace detection can be applied to find drugs and other illicit substances, which we class generally as contraband, although there are of course cases when one might be interested in such sampling techniques for non-contraband material. Screening may be useful for instance in quality assurance of organic foods and agriculture, where residual quantities of pesticides or additives can be measured to control the products as well as pharmaceutical and other products, or in sampling traces of volatile organic compounds near factories suspected of pollution, or the like.

### Manual Operation

Presently most sampling is done manually by more or less skilled operators using cotton or similar material swabs. Such procedures can be influenced by human error, attitude, skill, and materials preparation, therefore limiting the quality of performance and introducing an inevitable element of error into the process.

### Automation of Sampling

There are many systems aiming to partially or completely automate the sampling procedure in order to ameliorate aforementioned limitations and improve the efficiency of the procedure while also providing lower cost of operation. Amongst others, the “Ornath procedure” proposes certain sampling techniques based on a general procedure as follows: Enclose the item in a hermetically closed enclosure; blow short pulses of compressed and possibly heated air using nozzles that are pointed directly or indirectly at the item; oscillate the items using external means; blow air to increase the pressure inside the screened items; decompress the enclosure to exhale the air around the item as well as the air found inside it; draw air from the enclosure, containing traces to be analyzed, through a collection device such as filter media; send the traces on for analysis by ETD.

However there remains a long felt need for an automated system of high collection efficiency able to collect both vaporous and particulate samples. In addition, the success depends on the free flow of air around and within the baggage or cargo of interest. However, some of the most challenging targets for screening come packaged to some extent or other at the screening point. Shrink-wrapped packages that do not allow easy access to the items of interest are for instance difficult to analyze since the wrapping may be gastight; sealed containers may likewise be used to store items to evade detection by ‘sniffing’ methods such as those described above.

### BRIEF SUMMARY

I an aspect of the claimed invention, a device for precollection and preconcentration of solids and vapors in a fluid stream comprising: a chamber into which said fluid stream is entrained; a constricting bottleneck in the path of said fluid stream tending to increase the velocity thereof; a barrier plate at the exit from said bottleneck adapted to force said fluid stream to rapidly change direction; and vapor collection means disposed upon said barrier plate, wherein both solids and vapors are collected from said fluid stream.

It is further within provision of the invention wherein said barrier plate is porous.

It is further within provision of the invention wherein said vapor collecting means comprises material having chemical affinity for substances of interest.

It is further within provision of the invention wherein said material is phenyl-methyl polysiloxane.

It is further within provision of the invention wherein said vapor collection means are selected from the group consisting of zeolite and silica.

It is further within provision of the invention wherein barrier plate comprises a rough surface tending to cause particles and vapor to adhere thereto.

It is further within provision of the invention further wherein an atomized fluid is introduced into said fluid stream.

It is further within provision of the invention wherein a relatively high electric potential is introduced into said fluid stream so as to ionize elements of said fluid stream.

It is further within provision of the invention providing an electric potential to said barrier plate tending to attract said ionized elements.

In another aspect of the invention to disclose a method for precollection and preconcentration of solids and vapors in a fluid stream comprising steps of:
constricting said fluid stream in a bottleneck tending to increase the velocity thereof;
placing a barrier plate tending to force said fluid flow to rapidly change direction;
collecting solid particles on said barrier plate;
collecting vapor using vapor collection means disposed upon said barrier plate;
wherein both solids and vapors are collected from said fluid stream.

It is further within provision of the invention wherein said barrier plate is porous.

It is further within provision of the invention wherein said vapor collecting means comprises material having chemical affinity for substances of interest.

It is further within provision of the invention wherein said material is phenyl-methyl polysiloxane.

It is further within provision of the invention wherein said vapor collection means are selected from the group consisting of zeolite and silica.

It is further within provision of the invention wherein barrier plate comprises a rough surface tending to cause particles and vapor to adhere thereto.

It is further within provision of the invention further wherein an atomized fluid is introduced into said fluid stream.

It is further within provision of the invention wherein a relatively high electric potential is introduced into said fluid stream so as to ionize elements of said fluid stream.

It is further within provision of the invention providing an electric potential to said barrier plate tending to attract said ionized elements.

Another aspect of the present invention provides a device for screening packed cargo comprising at least one gas input hose adapted to force a working fluid into said packed cargo; and at least one gas exhaust hose adapted to exhaust said working fluid from said packed cargo; whereby packed cargo is penetrated by said working fluid and exhausted for purposes of trace analysis.

It is further within provision of the invention to vibrate said packed cargo for purposes of dispersing more trace material from said cargo into said fluid stream.

It is further within provision of the invention wherein said trace analysis comprises material having chemical affinity for substances of interest.

It is further within provision of the invention wherein the pressure in said input hose and said output hose is controlled to create a predefined profile of pressure vs. time.

It is further within provision of the invention wherein the temperature of said fluid is controlled for purposes of entraining more trace material from said cargo.

It is further within provision of the invention wherein said fluid is selected from the group consisting of: air, nitrogen, argon, helium, hydrogen, oxygen, carbon dioxide, trace-reactive molecules, and combinations thereof.

It is further within provision of the invention further wherein an atomized fluid is introduced into said fluid stream.

It is further within provision of the invention wherein a relatively high electric potential is introduced into said fluid stream so as to ionize elements of said fluid stream.

It is further within provision of the invention wherein said exhaust and input hoses are provided with puncturing means adapted to puncture gastight coverings of said cargo.

It is further within provision of the invention wherein extant apertures of said packed cargo are used for purposes of input and exhaust of said fluid.

In a further aspect of the invention, method for screening packed cargo comprising steps of:
forcing a working fluid into said packed cargo by means of at least one gas input hose 103; and,
exhausting said working fluid from said packed cargo by means of at least one gas exhaust hose 104;
whereby packed cargo is penetrated by said working fluid and exhausted for purposes of trace analysis.

It is further within provision of the invention to further providing adapted to vibrate said packed cargo for purposes of dispersing more trace material from said cargo into said fluid stream.

It is further within provision of the invention, wherein said trace analysis comprises material having chemical affinity for substances of interest.

It is further within provision of the invention, wherein the pressure in said input hose and said output hose is controlled to create a predefined profile of pressure vs. time.

It is further within provision of the invention, wherein the temperature of said fluid is controlled for purposes of entraining more trace material from said cargo.

It is further within provision of the invention, wherein said fluid is selected from the group consisting of: air, nitrogen, argon, helium, hydrogen, oxygen, carbon dioxide, trace-reactive molecules, and combinations thereof.

It is further within provision of the invention, wherein an atomized fluid is introduced into said fluid stream.

It is further within provision of the invention, wherein a relatively high electric potential is introduced into said fluid stream so as to ionize elements of said fluid stream.

It is further within provision of the invention, wherein said exhaust and input hoses are provided with puncturing means adapted to puncture gastight coverings of said cargo.

It is further within provision of the invention, wherein extant apertures of said packed cargo are used for purposes of input and exhaust of said fluid.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1 illustrates an Impactor with vapor collecting capability;

FIG. 2 illustrates porous silicon;

FIG. 3 illustrates a Hybrid Impactor.

FIG. 4 illustrates an embodiment of the invention utilizing input and output hoses penetrating a plastic sheath;

FIG. 5 illustrates an embodiment utilizing input and output hoses attaching to preexisting apertures in a shipping container.

### DETAILED DESCRIPTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for providing a system and method for sample precollection and preconcentration (as well as screening cargo).

In the following detailed description, numerous specific details are set forth in order to provide a thorough understanding of embodiments of the present invention. However, those skilled in the art will understand that such embodiments may be practiced without these specific details. To justly and entirely describe renditions of each embodiment may not yield full reportage of underlying concepts. Thus we may generally articulate that not all embodiments are necessarily described herein, but that the concepts underlying the invention are fully disclosed.

Reference throughout this specification to “one embodiment” or “an embodiment” means that a particular feature, structure, or characteristic described in connection with the embodiment is included in at least one embodiment of the invention.

The term ‘plurality’ refers hereinafter to any positive integer (e.g, 1, 5, or 10).

The term ‘contraband’ refers hereinafter to any material that may be of interest to law enforcement, immigration, border control, or other entities, including explosives such as nitrocellulose, smokeless powder, dynamite, gelignite, trinitrotoluene, C-4, HMX, PETN, RDX, Black powder, ANFO, Cheddites, Oxyliquits, Panclastites, Sprengel explosives, Armstrong's mixture, Ammonal, Acetone peroxide, Alkali metal ozonides, Ammonium permanganate, Ammonium chlorate, Azidotetrazolates, Azo-clathrates, Benzoyl peroxide, Benzvalene, Chlorine azide, Chlorine oxides, Copper(I) acetylide, Copper(II) azide, Cumene hydroperoxide, Cyanogen azide, Diacetyl peroxide, Diazodinitrophenol, Diazomethane, Diethyl ether peroxide 4-Dimethylaminophenylpentazole, Disulfur dinitride, Ethyl azide, Explosive antimony, Fluorine azide, Fluorine perchlorate, Fulminic acid, Hexamethylene triperoxide diamine, Hydrazoic acid, Hypofluorous acid, Lead azide, Lead styphnate, Lead picrate, Manganese heptoxide, Mercury(II) fulminate, Mercury nitride, Methyl ethyl ketone peroxide, Nitrogen trichloride, Nitrogen tribromide, Nitrogen triiodide, Nitroglycerin, Nitrotetrazolate-N-oxides, Octaazacubane, Pentazenium hexafluoroarsenate, Peroxy acids, Peroxymonosulfuric acid, Selenium tetraazide, Silicon tetraazide, Silver azide, Silver acetylide, Silver fulminate, Silver nitride, Sodium azide, Tellurium tetraazide, tert-Butyl hydroperoxide, Tetraamine copper complexes, Tetraazidomethane, Tetrazene explosive, Tetranitratoxycarbon, Tetrazoles, Titanium tetraazide, Triazidomethane, Xenon dioxide, Xenon oxytetrafluoride, Xenon tetroxide, Xenon trioxide; radioactive materials such as plutonium 239, polonium 210, technetium 99, Selenium 79, tin 126, zirconium 93, cesium 135, palladium 107, iodine 129, curium 244, Francium, Uranium, Thorium, Plutonium, Radium, Neptunium, californium 250, actinium 227, and cesium 245; proscribed, prohibited, illegal or otherwise suspect drugs and medicaments such as Heroin, Cocaine, Marijuana, Barbiturates, Novril, Digitalin, Ephemerol, Gambutrol, Inoprovaline, Methamphetamines, Neodextraline, Placiden, Prexilin, Qualex, Semuta, MDMA, Fukitol, and Triopenin; poisons such as Anticholinergics, haldol, risperidone, Atropine, scopolamine, Beta-Blockers, Propranolol, Sotalol, Caffeine, Xanthines, Cyanideethylene, glycolbenzodiazepines, barbiturates, Hydrofluoric acid, Iron, mercury, lead, boron, arsenic, cadmium, arsenic, Isoniazid Magnesiummethanol, Nicotine, opioids, Organophosphates, paracetamol (acetaminophen), Thallium, warfarin, Verapamil, and Diltiazem; rare elements and materials such as gold, iridium, neodymium, diamond, platinum, astatine, Francium, Technetium, and Promethium; and other materials that may be of interest to such bodies.

As should be clear to one skilled in the art, vapor having telltale traces of materials of interest will form in the air or fluid surrounding an object due to processes of evaporation, diffusion, sublimation, friction, and the like. This will occur to varying degrees in accordance with the vapor pressure of the substance(s) involved. Some substances have a very low vapor pressure, and while vapor always forms to an extent increasing with exposure time and temperature, the amount of evaporated material can be so minute as to be very difficult and identify. For such materials, it may be the case that the detection by detecting docile minutes solid (or liquid), the particles are from the surfaces by means of mechanical forces, such as vibration, friction, etc. Such particles, once free of the surface is dispersed, may be of extremely low weight, and will for a long time before to float in the absence of air currents in the presence of currents, they can be suspended in some cases indefinitely.

As mentioned in the background section, the manual or automated process of screening baggage and cargo items for the presence of explosives is a common procedure in transportation security procedures, cargo transport, package dispersal, mail operations, passenger movement, and other situations. The invention is associated with processes and devices for screening for substances items of interest using trace detection by means of collecting traces of vapor and particles emitted by substances of interest, due to processes of evaporation, diffusion, friction, and the like.

As will be clear to one skilled in the art, vapor having telltale traces will form in the air or fluid surrounding an object due to the aforementioned processes of evaporation, diffusion, sublimation, friction, and the like. This will occur to varying degrees in accordance with the vapor pressure of the substance (s) involved. Some substances have a very low vapor pressure, and while vapor always forms to an extent increasing with exposure time and temperature, the amount of evaporated material can be so minute as to be very difficult and identify. For such materials, it may be the case that the detection by minute solid (or liquid) particles released from the object surfaces by means of mechanical forces, such as vibration, friction, etc is one potential route for detection. Such particles, once free of the surface, may be widely dispersed, and may further be of extremely low weight in which case they may float for a long time before settling in the absence of air currents. In the presence of currents, they can be suspended in some cases indefinitely.

As outlined above, both vapor and particles of various substances of interest (such as drugs, explosives, radioactive materials, poisons, rare elements and the like) can form during the concealment of explosives in IEDs (Improvised Explosive Device) that terrorists prepare and that security agencies are tasked with foiling or as contamination during the concealment.

Explosive detection often uses the ubiquitous mentioned vapor and/or particle diffusion of matter to locate and defeat or prevent the surreptitious thereof by terrorists, smugglers or other inimical entities. After collection, traces may be detected by any of the known methods for such purposes. Explosive trace detectors for instance are known and designed to detect and identify the presence of explosive substances even in very small trace amounts.

Efficient collection of traces is obviously of crucial importance for the success of the screening process. Even if the detector is exceedingly sensitive, the success of the entire process still depends on a certain minimum amount of sample being collected and placed in contact with the detector.

As will be appreciated by one skilled in the art, sampling efficiency may be quantified as the fraction of the total amount of trace elements in a sampled volume that is actually brought to the detector. The more efficient the sampling system, the greater a fraction of all of the existing trace material is made available for analysis by the detection means.

Trace analysis can be employed as implied above for detection of drugs and other illegal substances, poisons, valuables, radioactive substances, explosives, incendiaries, controlled substances, and the like. Screening may also be useful in other cases. For example, in quality control of produce, organic food, farming products, flowers and the like, regulatory agencies or other entities may be interested in measurements of pesticides or additives to the products. Pharmaceutical and other products can be controlled for contamination and purity by such methods as well. Similarly when samples contain traces of pollutants, volatile organic compounds, and other substances near of factories suspected of contamination, this may raise a red flag with regulatory agencies or the like.

Currently, samples are often manually obtained by workers using wands tipped with cotton swabs or similar material on the ends adapted to absorb traces from materials of interest. Such methods may be affected by human error, attitude, skill, and processing technology, thus limiting the quality of detection and introducing an inevitable element of error into the process.

To address such, there have been many systems introduced adapted to partially or fully automate the sampling process in an attempt to improve the limitations and the efficiency thereof. Similarly, certain systems aim to providing lower operating costs.

One popular approach involves enclosing the object (or bag) to be checked in a hermetically sealed housing, blowing short pulses of compressed and possibly heated air into the housing using nozzles , possibly shaking the items with external means, increasing the pressure within the housing, decompressing the housing, drawing air therefrom, and analyzing the traces thus collected by entrainment in collection media such as filter paper or the like. Such traces are sent for trace analysis by the detectors of the device or by means of offsite detection systems. There thus remains a long-standing need for an automated system having a high filtration efficiency for both vapor and collect particulate samples.

The claimed invention relates to efficient methods for particle collection. The claimed invention s suitable for screening baggage and cargo for the presence of explosives, contraband, etc. as well as detection of trace pollutants, pesticides, and other materials that may be of interest in different situations as suggested above. As well as to screening packaged and/or wrapped objects for substances of interest by means of trace detection. This includes methods and devices for collecting traces of vapor and particles suitable for detecting substances of interest, based on particles and/or vapor emitted through evaporation, diffusion, sublimation, friction, and the like.

Simple trace collection using filter media has been used effectively in particular for particulate matter, although devices that collect vapor at least in some measure have been proposed. Collection of vapor-phase traces and residuals in air has been used widely to clean the air that is exhausted from industrial processes, preventing dangerous chemicals and pollutants from being released into the atmosphere. Techniques used include scrubbers, electrostatic filters and similar technologies. All are intended to allow clean air to pass and remove the pollutants.

But the aim of the current technology is different: for purposes of the invention, collection and preconcentration of traces must be accomplished with a view to facilitation of further analysis.

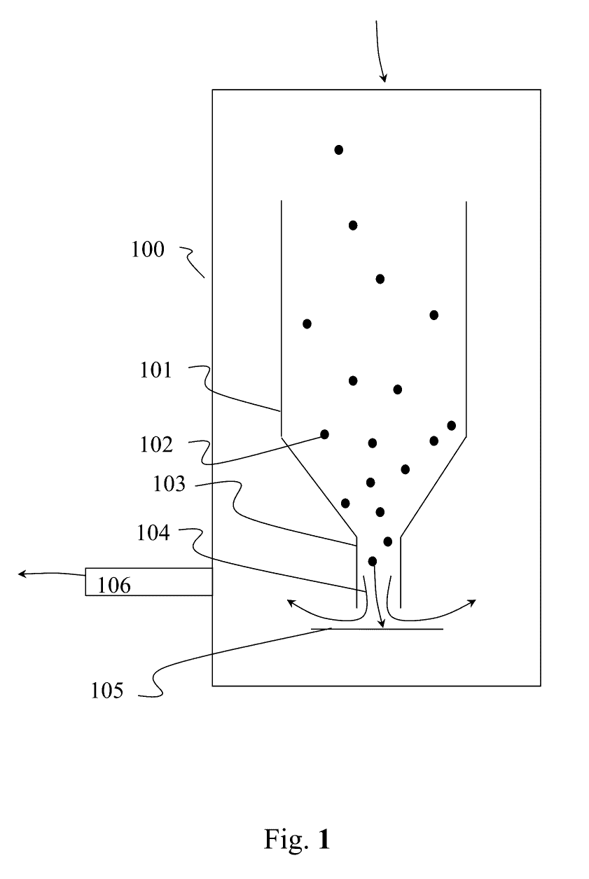

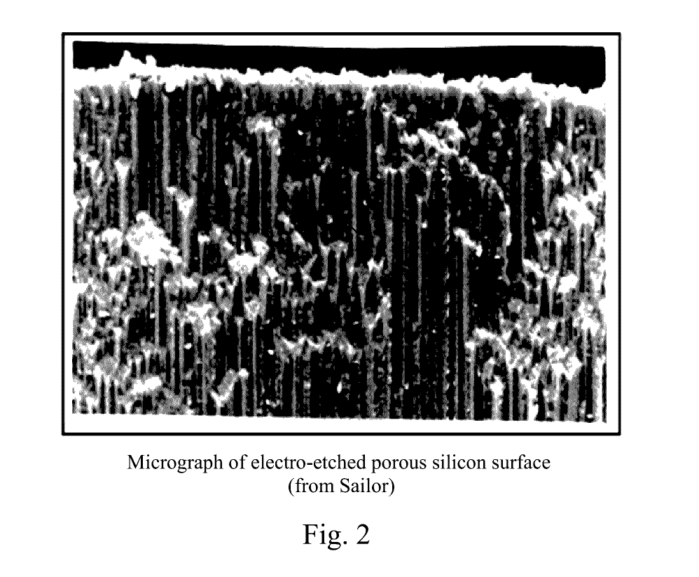

The hybrid impactor of the invention is similar to the impactor shown in FIG. 1. Here the impactor 100 consists of a cylindrical body 101 having some amount of forced air flow through it. The restriction section 103 tends to increase the fluid velocity through this section, causing the particles 102 entrained in the air flow 104 to impact the impact plate 105. The impact plate 105 is preferably made from a porous absorbing surface such as porous silicon as shown in FIG. 2.

The impactor (FIG. 1) is a device in which the air is accelerated for instance in the throat section 103, and some of the entrained particles are thereby made to impact a surface 105. As one sees in the figure, the air has to change its flow direction 104 abruptly and is drawn out through side openings. The particles that are carried in the air cannot change direction as fast as the air molecules due to their greater momentum and they will therefore in many cases impact the surface 105, where they are collected. A characteristic of this type of impactor is that it can collect particles but not vapor (since molecules are small enough to travel with the airflow.) Air enters the device at the top and exits out the exhaust tube 106.

Prior art collecting substrates for impactors include paper and similar materials. These are often made of somewhat rough material to improve retention of the particulate matter.

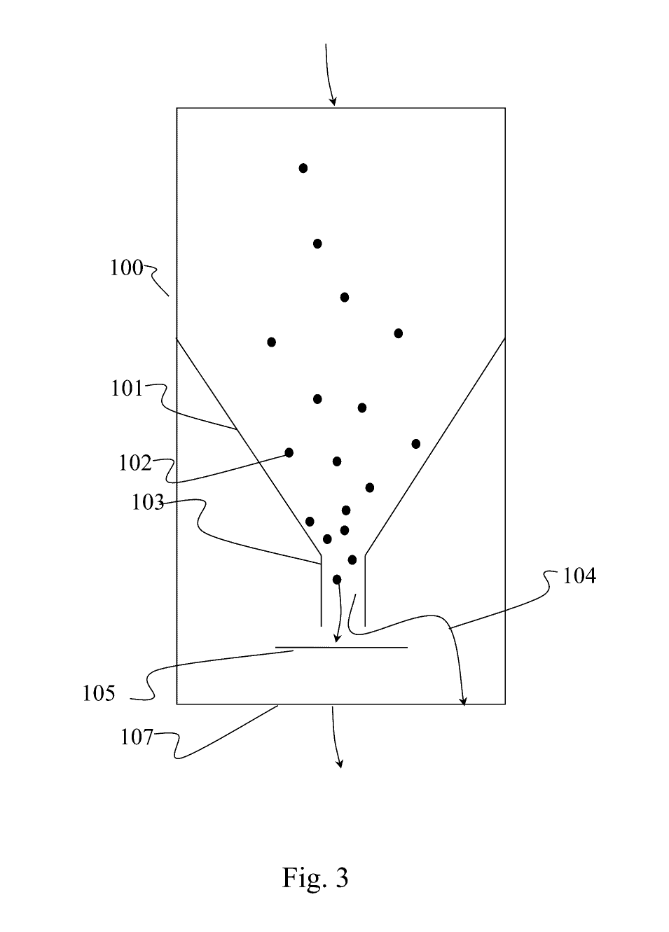

The current invention introduces a hybrid impactor using a similar arrangement to that shown in in FIG. 1, but adapted also to collect vapors in the air, hence the terminology hybrid impactor due to its ability to collect both particles and vapor traces. As evident in FIG. 3, instead of using a side exhaust tube, the device exhausts flow out the bottom end 107. The vapors may now be collected using filter media disposed in the bottom exit 107 of the device and/or in the impactor 105.

For these purposes the invention employs collection media (these being the collection substrate and/or collection surface) having superior absorption (preferably selective absorption) capability such as porous silicon. (See porous silicon in FIG. 2.)

The collection media can also be made to collect vapor in the following ways:

Firstly, vapor collection may be achieved by adding absorbing material on the upper surface of collection plate 105 (the surface that faces the flow) by use of binding powders of absorbing materials such as zeolite, silica, etc.

Second, vapors may be collected by adding absorbing material that has chemical affinity to the relevant substances (of the traces of interest) such as phenyl-methyl polysiloxane (this being used also as retention material for gas chromatography due to its affinity for many common substances).

A third technique involves making the collection media porous, such as to allow part of the air to pass through to the other side. In such cases, the collection substrate is made to have absorption capability by incorporation into it of absorbing material such as zeolite, silica and others as mentioned above. FIG. 3 shows such an arrangement. Air containing traces of materials of interest such as contraband, is accelerated against the target surface 105, and most of the air goes around the collecting substrate while the heavier particles are trapped on the surface. Part of the air passes through the substrate medium 105, and vapor and very small particles are then trapped in it.

After completing the collection cycle, the substrate can be heated to release the trace material in gaseous form, or bathed in a solvent to release the material in a liquid. The extract can be then submitted for analysis by a trace detector such as a mass spectrometer or the like as will be clear to one skilled in the art.

The operation of the device may be enhanced by injection of liquid drops into the sampling volume. In this embodiment, a cloud containing liquid solvent droplets in suspension is injected into the air flow to trap vapor and particles by means of adhesion and adsorption/absorption of vapor and particles to the liquid droplets. The liquid droplets are then accelerated by the air flow and collected on the aforementioned hybrid impactor device.

It is within provision of the invention that the liquid drops be ionized to provide an additional mechanism for electrostatic trapping particles.

Charged drops can also be easily collected on a collection substrate by means of applying a suitable electric potential to the substrate as will be clear to one skilled in the art.

Enhancement of the hybrid impactor efficiency may be effected by electrostatic charging as mentioned, even without use of liquid droplets. The air going into the device can be sent on a path near (for example) a needle bearing a high electrical voltage, to charge the vapor and solid particles passing nearby.

As with the case of droplets, these charged particles can also be better collected on the collection substrate when a suitable potential is applied to it.

The carrier gas used for transport of the vapor and solid particles of interest may be air, argon, nitrogen, or other gas or gas mixture as will be clear to one skilled in the art. Where air is used as a primary carrier (for example due to sampling in areas of human activity such as airport scanners) the air can subsequently be selectively replaced by another gas such as carbon dioxide or nitrogen that, as will be appreciated by those skilled in the art, may confer special advantages. The carrier fluid can also be a gas containing a certain amount of solvent vapor or a suspension of droplets that can help in the carrying or subsequent processes such as DMSO, acetone, IPA (isopropyl alcohol), etc.

A time-worn method for explosive detection employs dogs to detect the almost inevitable diffusion of vapor and/or particles from materials of interest, to locate and identify these materials. Other more modern means involve use of analytical chemistry, whereby after collecting traces, these are analyzed by means of techniques including IMS, MS, and GC amongst others. Bespoke detectors have been developed to detect and identify the presence of specific compounds such as explosives in trace amounts.

Vapor as mentioned is in most cases released to some extent by substances of interest by the aforementioned nearly inevitable processes of evaporation and diffusion. As mentioned this will occur to varying degrees in accordance with the temperature and resulting vapor pressure of the substance(s) involved. Some substances have a very low vapor pressure and thus the amount of vapor produced will be exceedingly low. Others may sublime upon heating to a certain extent varying with varying exposure time and temperature. In either case, it should be appreciated that the amount of vapor-phase material can be so small that it is difficult to detect and identify.

For such materials, it may be the case that detection by means of minute solid (or liquid) particles discharged from material surfaces (by mechanical forces such as vibration, friction, etc.) may be effective. Such particles, once free of the surface will tend to disperse by dint of Brownian motion, diffusion, and ambient air currents. Because the particles are often extremely light weight and have potentially large surface area, they can be suspended for long periods of time, in the presence of sufficient air flow, such particles can remain suspended indefinitely.

As stated above, both vapor and particles of various substances of interest (such as drugs, explosives, radioactive materials, poisons, rare elements and the like) will generally be released from materials and is usually present in the form of an invisible “cloud” having telltale chemical composition of elements of interest.

In this context it should be clear that efficient collection of material traces by sampling the air around objects is crucial for the success of the screening process. As sensitive as a given detector may be, success in identifying materials of interest still depends largely on the amount of sample provided to the detector, which will generally have some lower detection limit. The criterion of sampling efficiency is thus of paramount importance; the more efficient the sampling system, the larger a fraction of all existing trace material will be made available for analysis by the detection means at hand. As will be appreciated, well packed and/or extremely stable or inert substances necessitate the detection of particles or vapor in extremely dilute mixtures, a situation exacerbated by requirement in some situations for reduction of the sampling volume and/or decreased sensitivity of (for example) portable equipment as opposed to lab equipment.

As already mentioned, trace analysis may be useful for detecting concealed explosives and drugs, but can also be used for other classes of materials and different detection goals. Trace detection can be useful for example in quality control for the agricultural products, in order (for example) to identify residues of pesticides or preservatives. In pharmaceutical and other contexts, such means may prove to be useful, for example to detect impurities, component concentrations, and other entities. Also pollution near factories can be monitored, identification of clothing or other items for criminal identification and/or missing persons rescue may be facilitated, and a variety of other applications are possible to be understood by a person skilled in the art.

The method and device of the invention are aimed at effective collection and preconcentration of traces of contraband in air or other fluids. Collection and preconcentration are necessary and useful for a number of applications including explosive detection and identification.

The importance of effective sampling methodology for successful detection of minute concentrations of substances such as explosives, drugs, and sundry molecules cannot be stressed highly enough; the concentrations involved may be exceedingly small, comprising parts per billion or less, such that without these steps the possibility of detection becomes difficult if not impossible without resort to extreme means.

Efficient collection and preconcentration of trace material is further important since (for example) in explosive trace detection (ETD) at airports, only a very small amount of air from each passenger can be sampled and analyzed due to the high passenger traffic rates that must be handled.

Although selected embodiments of the present invention have been shown and described, it is to be understood the present invention is not limited to the described embodiments. Instead, it is to be appreciated that changes may be made to these embodiments without departing from the principles and spirit of the invention, the scope of which is defined by the claims and the equivalents thereof.

There are many systems extant for partial or full automation of the sampling approaches, to improve the efficiency and the limits of the process. Often such methods include enclosing the object to be checked in an airtight enclosure, having short pulses of short compressed air (possibly heated) and/or mechanical vibration/oscillation of the cargo or other container being checked, by use of vibratory motors or the like. If the container is somewhat airtight, the introduction of air into the chamber, thereby affecting the pressure within, and using a step of decompression thereafter, can often be efficacious. The decompression may be accomplished by removing some amount of the air from the chamber, said removed air then being analyzed for traces of substances of interest in a manner consistent with the art as will be clear to one skilled therein. As will be appreciated the variation of pressure over time may be of use in causing materials to “shed” telltale particles and/or vapor.

The success of the methods outlined above depends of course upon the free flow of air to and from the object of interest. Within the baggage or cargo of interest lies the contraband to be detected. However, some of the more challenging targets for screening come packaged in more or less airtight packaging. Shrink wrap packages for example abound, for which access to the elements of interest to be analyzed is hampered because the packaging may be gas-tight or ‘gas proof’ to some extent. Sealed shipping containers can also be used to store items in order to avoid detection by the various “sniffing” techniques such as described above.

The method and apparatus of the invention are directed to efficient collection and concentration of trace amounts of contraband packed in and/or wrapped in airtight or nearly airtight containers, plastic wrapping, or the like . As expounded at length above, collection and concentration are useful and/or crucial for a number of applications including explosive detection and identification.

For purposes of ‘sniffing’ airtight containers, the invention makes use of hoses to introduce and exhaust gases to such containers, if need be by means of puncturing the gastight envelope surrounding the packed objects. Opening such containers entirely for individual screening of packages is often not tenable as it is time consuming and requires large investments in equipment and personnel, in addition to potentially invoking insurance liability and/or other thorny issues.

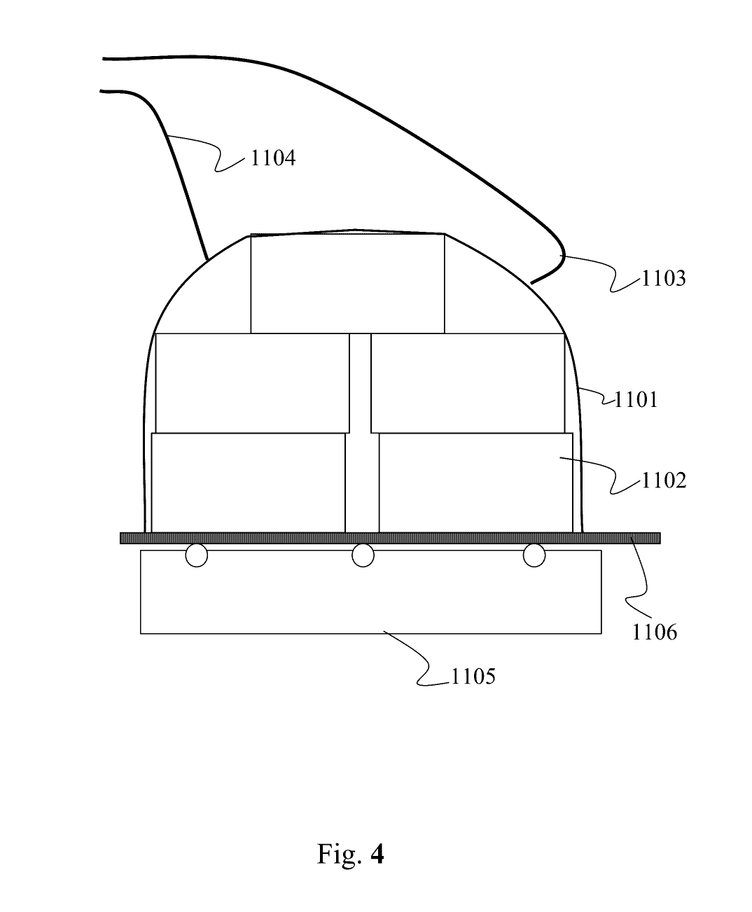

FIG. 4 illustrates an example embodiment of the invention. Here a pallet 1106 of material being shipped has been wrapped with airtight plastic sheeting 1101. For purposes of ‘sniffing’ the cargo 1102 within, normal methods are powerless since the sheeting 1101 forms an airtight or nearly airtight barrier preventing exudation of vapor or particles from the cargo. Therefore two hoses are used to puncture the barrier; an inlet hose 1104 forces a carrier gas such as filtered air into the membrane formed by the sheeting 1101, and an outlet hose 1103 collects gas from within the membrane. The pallet may furthermore be located upon a movable ‘shaker’ 1105 adapted to vibrate or shake the entire pallet and its contents, an action tending to release more vapor and small particles from the cargo 1102.

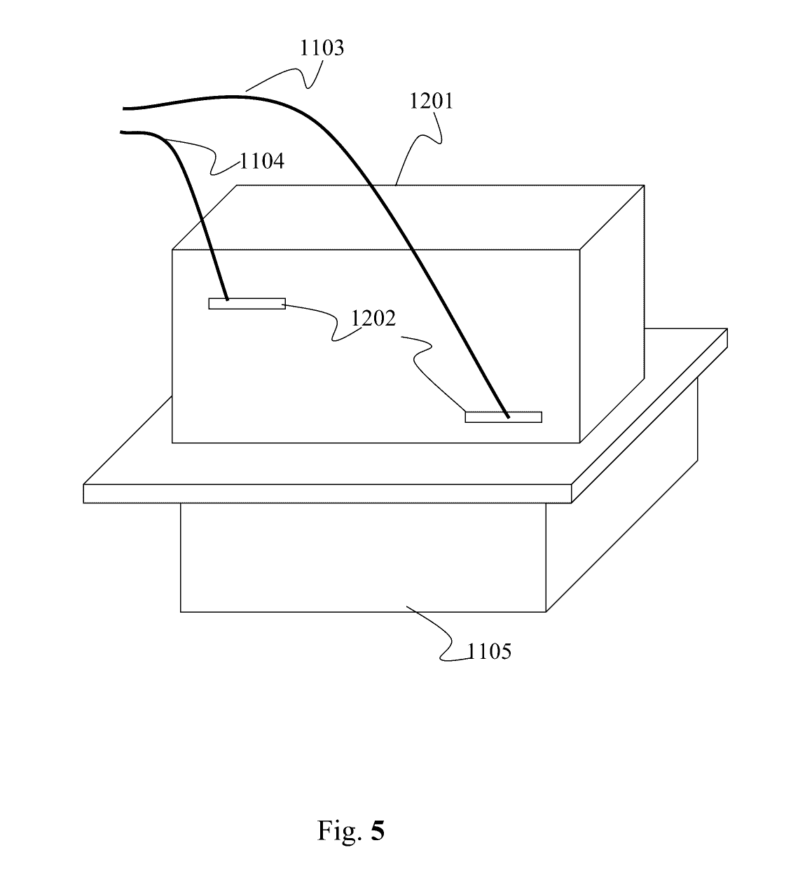

FIG. 5 illustrates an alternative embodiment useful for situations when one or more holes or apertures are already extant in a package or container 1201. For purposes of ‘sniffing’ the items within container 1201, two hoses 1103,1104 are used to provide a carrier gas entering and exiting by means of extant apertures 1202. These hoses may have nozzles adapted to puncture various wrappings or coverings. An inlet hose 104 forces a carrier gas such as filtered air into the container, and an outlet hose 103 collects gas from within the container. The container may furthermore be located upon a movable ‘shaker’ 1105 adapted to vibrate or shake the entire pallet and its contents, an action tending to release more vapor and small particles from the cargo within the container 1201.

As will be appreciated by one skilled in the art, the invention is substantially different from previous technology, as there is no use of an adaptive housing, but rather the invention employs the use of the existing packing case with the special nozzles or hoses mentioned above. Amongst other advantages it will be appreciated that the atmosphere within the packages (also known as “headspace”) will have had ample time to come into equilibrium with the contents of the packages and thus any trace materials will be present in relatively high concentration.

The nozzles of the hoses 1103,1104 as mentioned may be fitted with special ejection nozzles that can be used to puncture such coverings as shrink film. These nozzles are connected via the hoses to a source of compressed air or other fluid, and means for exhausting such. Pulses of air or other working fluid may be employed, for example using quick pressure pulses. The fluid used may be optionally heated to further encourage entrainment of traces into the fluid stream; as will be appreciated the vapor pressure of most materials will increase with temperature.

The nozzles are used to penetrate the packaging, obviating the need for removal of the packing case. The nozzles are adapted so as not to pierce the packaging of various packages within the envelope or wrapping of the pallet or packages, but may be employed for such if desired. Alternatively, special penetrating nozzles may be used if required for this function.

The nozzle uses various means as will be clear to one skilled in the art specific to provide hermetic seal with the wrapping or packaging material, such that air does not escape easily from the puncture created by introducing the nozzle.

The exhaust tube(s) are preferably mounted using a generally hermetic attachment mechanism, at a different point on the shrink film from the entrance hose. This will preferably be as far as possible from the entrance hose, and at a lower position than it. This tube serves for the exhaust gas and connects to one or more trace collection devices. Such may comprise the filter media, possibly having a pre-cleaner, and/or a preconcentrator device inline.

Compressed air pulses may by means of these hoses directly and/or indirectly displace particles and entrain such as well as vapor that will generally be present in the headspace.

Shaking the pallet allows airflow between packaged articles, and promotes the spread of particulate traces which can be found in these areas. Such particles will be released from the interior of the packing elements and entrained in the gas flow, for analysis downstream after exhaustion.

Higher than ambient pressure may be used in the inlet hose to introduced gas or other fluid into the internal volume of the packaged article, and any pressure lower than this may be enforced in the outlet hose, for example by use of vacuum.

As mentioned air is drawn through the exhaust hose(s) and directed to a collecting device. The collected sample is analyzed for detection of trace particle of interest.

The fluid used for entrainment can be gas such as CO₂, which may in some cases and for some traces have a better collection affinity. Air, which allows for a small amount of solvent vapor, may be substituted with any number of other gases which may be of help in entraining particulate matter and vapor from substances of interest. This gas may also contain small amounts of solvent vapor, or atomized droplets in suspension. For example the solvent substances DMSO, acetone or similar substance, have special affinity for explosive materials.

An external additional housing can be used to control the ambient pressure around the entire object being checked (including film or other wrapping). This will be of use to reduce forces on the shrink film and to prevent its excessive deformation or destruction, as internal pressure may be formed by applying an external pressure to the outside (on the package), to counter the pressure inside the packaging. Such an external housing also allows for decreasing the forces on the sealing mechanisms mentioned above for the nozzles.

Seagoing or other shipping containers may be inspected using a similar arrangement as shown in FIG. 5. There is no shrink wrapping in this case, but the container itself is the external enclosure and may well be airtight. The external enclosure will generally be closed , but the container has certain openings, some of a standard size, to allow the passage of air into and out of the container; these standardized openings may be employed for introduction and exhaustion of fluids for entrainment of trace materials, for example by use of nozzles adapted to mate thereto in a gastight fashion. Special ejection nozzles can be attached to one or more of these openings using sealing mechanisms obvious to one versed in the art.

The exhaust lines may be made preferably low compared to the inlet nozzles (and possibly wide to exploit rectangular container openings) , so as to provide a collection path involving some loss of height.

As before, an optional external housing, if necessary flexible, may be provided to generate an external overpressure and/or underpressure. Variation of the internal and external pressures by means of changing the inlet hose pressure, exhaust hose pressure, and possibly external housing pressure, may all be employed for purposes of entraining more trace material into the fluid stream. Traces which can be found on the outer surfaces of the container may also be collected when using the optional external housing.

If such an external housing is employed, the housing may in some embodiments be disposable and replaced after each test, or as needed. If any such need occurs (for example as indicated by use of a system checking enclosure airtightness and seal integrity, or by use of results of the trace detection system) the system must be cleaned thoroughly as remaining traces may cause false alarms can result in further studies. But if the housing is not necessary, it may not require such cleaning thus allowing simplification of the procedure. When an external housing is employed, exhaust pipes may not be required. Instead the air may be introduced through the holes and apertures to escape the carton. The air is then collected from the external enclosure, and analyzed.

In another embodiment, a similar process is used to select items packed in cartons for inspection without opening the cartons. For this case, special cardboard penetrating nozzles may be employed using pulsed jets of air within the package. This is possible in most cases, because packed articles are often separated from the carton walls by shock-absorbing material, and therefore the nozzle will not damage the goods in boxes but may be employed in the space between the member and the box walls.

As a further embellishment to the techniques stated above, the temperature of the carrier gas(es) introduced into and exhausted from the containers may be elevated so as to encourage vapourisation and mobility of particles, or decrease surface adhesion forces of materials within. Furthermore, it is within provision of the invention that the pressures of incoming and outgoing lines 1103, 1104 may be varied so as to increase the total pressure within the cargo or container, for example reaching pressures above ambient pressure, and/or to decrease the inlet hose pressure below ambient, to employ cycles of pressurization and depressurization, to control pressure of the outlet hose simultaneously so as to engender a predetermined pressure gradient between inlet and outlet, and the like. This may all potentially tend to encourage vapourisation and/or physical emission of particles from objects in the container or wrapping, thus facilitating detection of trace particles so emitted.

The operation of the device can be improved by the injection of liquid droplets into the sample volume. In this embodiment, a liquid solvent containing atomized droplets in suspension is injected into the air stream of the inlet hose, to collect vapor and particles by means of adhesion and adsorption/absorption of vapor and particles to the liquid droplets. The liquid droplets are then entrained into the air flow and collected by the outlet hose.

It is within the invention to make use of ionized liquid droplets to provide an additional mechanism for the electrostatic trapping of particles. High potentials may be introduced within the package as well for purposes of ionizing objects therewithin.

Use of such charged particles may render collection easier, as for example the outlet hose may be provided with an electric potential tending to collect ions formed by other parts of the system.

Enhancement of the hybrid impactor or other type of collector mechanism efficiency may be attained by electrostatic charging, as mentioned, without the use of liquid droplets. The air may for example pass a needle bearing a high electrical voltage tending to ionize atoms passing by.

As in the case of droplets, these charged particles may enhance collection by means of attracting vapor or other particles of interest.

The carrier gas for the transport of the solid particles and vapor of interest may be used air, argon, nitrogen, carbon dioxide, or another gas or gas mixture as will be understood as one skilled in the art are in the field. If not restricted to using air as a carrier (for example, when scanning in areas of human activity, such as airports) the air can be selectively replaced with another gas, such as carbon dioxide or nitrogen, that, as will be recognized by those skilled in the art, may possess special advantages . The carrier fluid may also contain an amount of solvent vapor, or a suspension of droplets, aiding in the procedure by absorbing molecules or materials of interest. Solvents of use may include DMSO, acetone, IPA (isopropyl alcohol), etc.

It is within provision of the invention to employ any useful working fluid to pump into the packed container for purposes of entraining traces of contraband for further analysis. For example air, nitrogen, argon, helium, hydrogen, oxygen, carbon dioxide, trace-reactive molecules, and combinations may be used. By ‘trace-reactive molecules’ we mean molecules or compounds that are adapted to react with traces of interest, which may facilitate further analysis.

1. A method for screening packed cargo comprising steps of:
forcing a working fluid into said packed cargo by means of at least one gas input hose; and
exhausting said working fluid from said packed cargo by means of at least one gas exhaust hose;
whereby packed cargo is penetrated by said working fluid and exhausted.

2. The method of claim 1 further said packed cargo for encouraging dispersing of trace material from said cargo into said fluid stream.

3. The method of claim 1 further comprising step of trace analysis by using at least one material having chemical affinity for substances of interest.

4. The method of claim 1 further comprising step of controlling the pressure in said input hose and said output hose.

5. The method of claim 5 further comprising step of creating predefined profile of pressure vs. time.

6. The method of claim 1 further comprising step of controlling temperature of said fluid encouraging dispersing of trace material from said cargo into said fluid stream.

7. The method of claim 1 wherein said fluid is selected from the group consisting of: air, nitrogen, argon, helium, hydrogen, oxygen, carbon dioxide, trace-reactive molecules, and combinations thereof.

8. The method of claim 1 further comprising step of introducing a relatively high electric potential into said fluid stream encouraging ionization of elements of said fluid stream.

9. The method of claim 1 further comprising step of puncturing gastight covering of said cargo.

10. The method of claim 1 wherein extant apertures of said packed cargo are used for purposes of input and exhaust of said fluid.

11. The method of claim 1 further comprising steps of:
constricting said fluid stream in a bottleneck tending to increase the velocity thereof;
placing a barrier plate tending to force said fluid flow to rapidly change direction; and
collecting solid particles on said barrier plate; collecting vapor using vapor collection means disposed upon said barrier plate.

12. The method of claim 11 further wherein said barrier plate is porous.

13. The method of claim 11 wherein said vapor collecting means comprises material having chemical affinity for substances of interest.

14. The method of claim 11 wherein barrier plate comprises a rough surface tending to cause particles and vapor to adhere thereto.

15. The method of claim 1 further comprising step of introducing an atomized fluid into said fluid stream.

16. The method of claim 8 further comprising step of providing an electric potential to said barrier plate tending to attract said ionized elements.
